(19) United States
(12) Patent Application Publication
(10) Pub. No.: US 2017/0090185 A1
(43) Pub. Date: Mar. 30, 2017

(54) OPTICAL SCANNING APPARATUS AND OPTICAL SCANNING OBSERVATION APPARATUS

(71) Applicant: OLYMPUS CORPORATION, Tokyo (JP)

(72) Inventor: Ken FUJINUMA, Tokyo (JP)

(73) Assignee: OLYMPUS CORPORATION, Tokyo (JP)

(21) Appl. No.: 15/377,009

(22) Filed: Dec. 13, 2016

Related U.S. Application Data
(63) Continuation of application No. PCT/JP2015/003001, filed on Jun. 16, 2015.

(30) Foreign Application Priority Data
Jun. 18, 2014 (JP) 2014-125470

(51) Int. Cl. G02B 26/10; G02B 23/24; A61B 1/06; A61B 1/07; A61B 1/00; A61B 1/04

(52) U.S. Cl. CPC G02B 26/103; A61B 1/00006; A61B 1/00009; A61B 1/00096; A61B 1/04; A61B 1/063; A61B 1/0684; A61B 1/07; A61B 1/00027; G02B 23/2469; A61B 1/00045

(57) ABSTRACT

Included are an optical fiber (11), a driver (21) that drives an emission end (11b) of the optical fiber (11), a current detector (55) that detects a current flowing in the driver (21), and a controller (31) that scans light emitted from the optical fiber (11) by controlling the driver (21) based on output of the current detector (55). The driver (21) comprises vibration elements (28a to 28d), ground terminals of the vibration elements (28a to 28d) are connected in common to the driver (21), and the current detector (55) detects the current at the power supply terminal of the vibration elements (28a to 28d).

### CROSS-REFERENCE TO RELATED APPLICATION

The present application is a Continuing Application based on International Application PCT/JP2015/003001 filed on Jun. 16, 2015, which in turn claims priority to Japanese Patent Application No. 2014-125470 filed on Jun. 18, 2014, the entire disclosure of these earlier applications being incorporated herein by reference.

### TECHNICAL FIELD

This disclosure relates to an optical scanning apparatus and an optical scanning observation apparatus that optically scan an object by vibrating an optical fiber.

### BACKGROUND

One known example of an optical scanning observation apparatus vibrates the emission end of an optical fiber to scan a beam of light from the optical fiber over an object and detects light that is reflected, scattered, or the like by the object or detects fluorescent light or the like generated by the object (for example, see JP 4672023 B2 (PTL 1)).

### CITATION LIST

### Patent Literature

PTL 1: JP 4672023 B2

### SUMMARY

An optical scanning apparatus according to this disclosure includes:

an optical fiber;

a driver configured to drive an emission end of the optical fiber;

a current detector configured to detect a current flowing in the driver; and

a controller configured to scan light emitted from the optical fiber by controlling the driver based on output of the current detector; wherein

the driver comprises a plurality of vibration elements;

ground terminals of the vibration elements are connected in common to the driver; and

the current detector detects the current at a power supply terminal of the vibration elements.

The current detector may include a current transformer.

The vibration elements may include a plurality of first vibration elements that vibrate the emission end in a first direction and a plurality of second vibration elements that vibrate the emission end in a second direction different from the first direction;

power supply terminals of the first vibration elements may be connected in common to the driver; and

power supply terminals of the second vibration elements may be connected in common to the driver.

The controller may control the driver based on output of the current detector at a time when an amplitude of the current is maximized.

The controller may control the driver so that a maximum value of the current detected by the current detector becomes constant.

Furthermore, an optical scanning observation apparatus includes:

the aforementioned optical scanning apparatus;

a photodetector configured to detect light obtained from an object by optical scanning with the optical scanning apparatus and to convert the light to an electrical signal; and

an image processor configured to generate an image based on the electrical signal output from the photodetector.

### BRIEF DESCRIPTION OF THE DRAWINGS

In the accompanying drawings:

FIG. 1 is a block diagram schematically illustrating the structure of an optical scanning endoscope apparatus according to Embodiment 1;

FIG. 2 is a schematic overview of the scope of the optical scanning endoscope in FIG. 1;

FIG. 3 is a cross-sectional diagram of the tip of the scope in FIG. 2;

FIG. 4A illustrates the vibration driving mechanism of the driver and the emission end of the optical fiber for illumination in the optical scanning endoscope apparatus in FIG. 1;

FIG. 4B is a cross-section along the A-A line in FIG. 4A;

FIG. 5 is a block diagram schematically illustrating the structure of the drive controller/resonance frequency detector in FIG. 1;

FIG. 6 is a flowchart illustrating operations of the optical scanning endoscope apparatus in FIG. 1;

FIG. 7A shows the amplitude of driving voltage to illustrate operations of the optical scanning endoscope apparatus in FIG. 1;

FIG. 7B shows the frequency of driving voltage to illustrate operations of the optical scanning endoscope apparatus in FIG. 1;

FIG. 7C shows laser output to illustrate operations of the optical scanning endoscope apparatus in FIG. 1;

FIG. 7D shows the waveform of output voltage to illustrate operations of the optical scanning endoscope apparatus in FIG. 1;

FIG. 7E shows the scanning trajectory of illumination light to illustrate operations of the optical scanning endoscope apparatus in FIG. 1;

FIG. 8A illustrates the typical frequency characteristics of impedance;

FIG. 8B illustrates the typical frequency characteristics of phase shift;

FIG. 9A is an expanded cross-sectional diagram illustrating the tip of the optical scanning endoscope apparatus according to Embodiment 2;

FIG. 9B is an enlarged perspective view of the driver in FIG. 9A;

FIG. 9C is a cross-sectional view perpendicular to the axis of the optical fiber 11 for illumination, illustrating a portion including the coils for generation of a deflecting magnetic field and the permanent magnet in FIG. 9B;

FIG. 10 is a flowchart illustrating operations of the optical scanning endoscope apparatus in FIGS. 9A to 9C; and

FIG. 11 is a block diagram schematically illustrating the main structure of an optical scanning endoscope apparatus according to Embodiment 3.

### DETAILED DESCRIPTION

Embodiments are described below with reference to the drawings.

### Embodiment 1

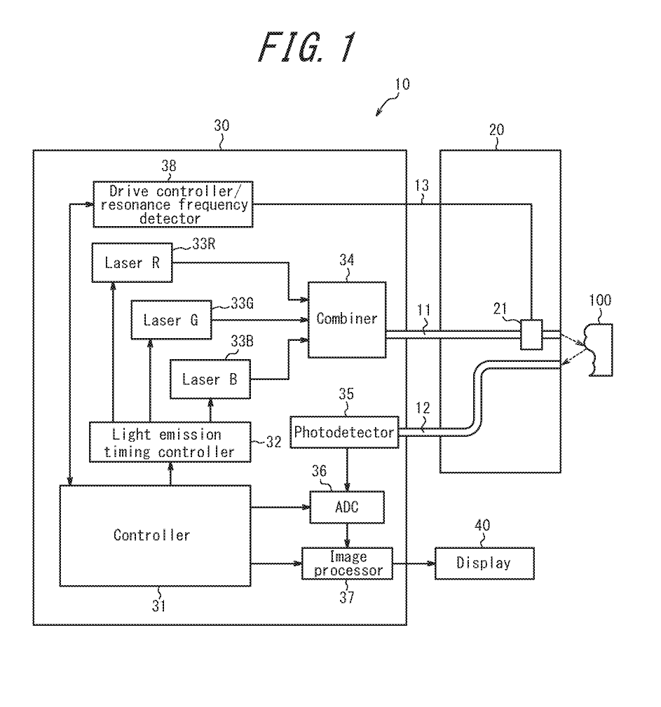

FIG. 1 is a block diagram schematically illustrating the structure of an optical scanning observation apparatus according to Embodiment 1. The optical scanning observation apparatus in FIG. 1 constitutes an optical scanning endoscope apparatus 10. The optical scanning endoscope apparatus 10 includes a scope (endoscope) 20, a control device body 30, and a display 40.

The control device body 30 includes a controller 31 that controls the optical scanning endoscope apparatus 10 overall, a light emission timing controller 32, lasers 33R, 33G, and 33B, and a combiner 34. The laser 33R emits red laser light, the laser 33G emits green laser light, and the laser 33B emits blue laser light. Under the control of the controller 31, the light emission timing controller 32 controls the light emission timing of the three lasers 33R, 33G, and 33B. For example, Diode-Pumped Solid-State (DPSS) lasers or laser diodes may be used as the lasers 33R, 33G, and 33B. The laser light emitted from the lasers 33R, 33G, and 33B is combined by the combiner 34 and is incident as white illumination light on an optical fiber 11 for illumination, which is formed by a single-mode fiber. The combiner 34 may, for example, be configured to include a dichroic prism or the like. The configuration of the light source in the optical scanning endoscope apparatus 10 is not limited to this example. A light source with one laser may be used, or a plurality of other light sources may be used. The lasers 33R, 33G, and 33B and the combiner 34 may be stored in a housing that is separate from the control device body 30 and is joined to the control device body 30 by a signal wire.

The optical fiber 11 for illumination extends to the tip of the scope 20. Illumination light incident on the optical fiber 11 for illumination via the combiner 34 is guided to the tip of the scope 20 and irradiated towards an object 100. At this time, the emission end of the optical fiber 11 for illumination is subjected to vibration driving by the driver 21. As a result, the observation surface of the object 100 is scanned in 2D by illumination light emitted from the optical fiber 11 for illumination. The driver 21 is controlled by the drive controller/resonance frequency detector 38 of the below-described control device body 30. Signal light, such as reflected light, scattered light, fluorescent light, and the like obtained from the object 100 by irradiation with illumination light is incident on the end face of an optical fiber bundle 12 for detection, which is formed by multi-mode fibers extending inside the scope 20, and the signal light is then guided to the control device body 30.

The control device body 30 further includes a photodetector 35 for processing signal light, an analog/digital converter (ADC) 36, an image processor 37, and a drive controller/resonance frequency detector 38. The photodetector 35 divides the signal light optically guided by the optical fiber bundle 12 for detection into spectral components and converts the spectral components into electric signals with a photodiode or the like. The ADC 36 converts the analog electric signals output from the photodetector 35 into digital signals and outputs the digital signals to the image processor 37. Based on information such as the amplitude, phase, and the like of vibration voltage applied by the drive controller/resonance frequency detector 38, the controller 31 calculates information on the scanning position along the scanning trajectory of laser illumination light and provides the information to the image processor 37. The image processor 37 sequentially stores pixel data (pixel values) of the object 100 in a non-illustrated memory based on the digital signals output by the ADC 36 and the scanning position information from the controller 31. After completion of scanning or during scanning, the image processor 37 generates an image of the object 100 by performing image processing, such as interpolation, as necessary and displays the image on the display 40.

In the above-described processing, the controller 31 synchronously controls the light emission timing controller 32, the photodetector 35, the drive controller/resonance frequency detector 38, and the image processor 37.

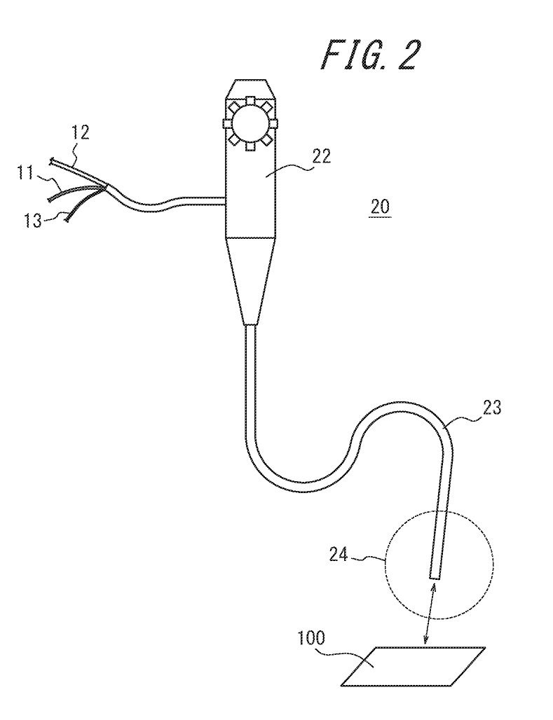

FIG. 2 is a schematic overview of the scope 20. The scope 20 includes an operation part 22 and an insertion part 23. The optical fiber 11 for illumination, the optical fiber bundle 12 for detection, and wiring cables 13 extending from the control device body 30 are each connected to the operation part 22. The optical fiber 11 for illumination, optical fiber bundle 12 for detection, and wiring cables 13 pass through the insertion part 23 and extend to a tip 24 (the portion within the dotted line in FIG. 2) of the insertion part 23.

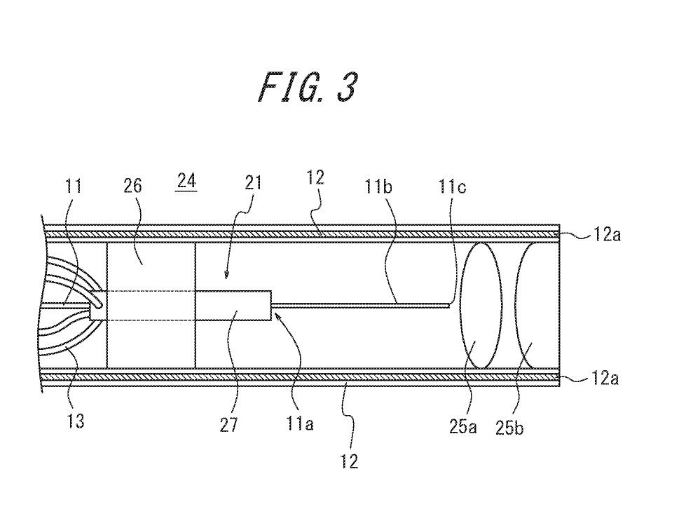

FIG. 3 is a cross-sectional diagram illustrating an enlargement of the tip 24 of the insertion part 23 of the scope 20 in FIG. 2. The tip 24 includes the driver 21, projection lenses 25a and 25b, the optical fiber 11 for illumination that passes through the central portion of the scope 20, and the optical fiber bundle 12 for detection that passes through the peripheral portion.

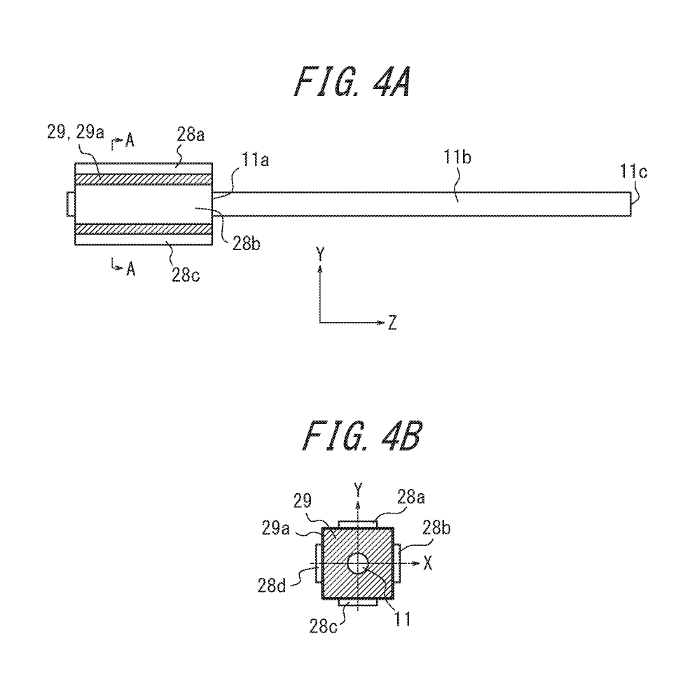

The driver 21 includes an actuator tube 27 fixed to the inside of the insertion part 23 of the scope 20 by an attachment ring 26, a fiber holding member 29 disposed inside the actuator tube 27, and piezoelectric elements 28a to 28d (see FIGS. 4A and 4B). The optical fiber 11 for illumination is supported by the fiber holding member 29, and the emission end 11b from the fixed end 11a by the fiber holding member 29 to the emission end face 11c is capable of oscillation. The optical fiber bundle 12 for detection is disposed to pass through the peripheral portion of the insertion part 23 and extends to the end of the tip 24. A non-illustrated detection lens is also provided at the tip of each fiber in the optical fiber bundle 12 for detection.

Furthermore, the projection lenses 25a and 25b and the detection lenses are disposed at the extreme end of the tip 24. The projection lenses 25a and 25b are configured so that laser light emitted from the emission end face 11c of the optical fiber 11 for illumination is roughly concentrated on the object 100. The detection lenses are disposed so that light that is reflected, scattered, refracted, or the like by the object 100 (light that interacts with the object 100), fluorescent light, or the like due to laser light concentrated on the object 100 is captured as signal light, concentrated on the optical fiber bundle 12 for detection disposed behind the detection lenses, and combined. The projection lenses are not limited to a double lens structure and may be structured as a single lens or as three or more lenses.

FIG. 4A illustrates the vibration driving mechanism of the driver 21 and the emission end 11b of the optical fiber 11 for illumination in the optical scanning endoscope apparatus 10. FIG. 4B is a cross-sectional diagram along the A-A line in FIG. 4A. The optical fiber 11 for illumination passes through the center of the fiber holding member 29, which has a prismatic shape, and is fixed and held by the fiber holding member 29. The four sides of the fiber holding member 29 respectively face the ±Y direction and the ±X direction when the optical axis of the optical fiber 11 for illumination in the fiber holding member 29 is in the Z direction, and directions that traverse the optical axis in a plane orthogonal to the optical axis and are orthogonal to each other are the Y direction (first direction) and the X direction (second direction). A pair of piezoelectric elements 28a and 28c for driving in the Y direction are fixed onto the sides of the fiber holding member 29 in the ±Y direction, and a pair of piezoelectric elements 28b and 28d for driving in the X direction are fixed onto the sides in the ±X direction.

The piezoelectric elements 28a to 28d each constitute a vibration element, and the ground terminals (one of the surface electrodes) of the piezoelectric elements are connected in common in the driver 21. For example, the ground terminals of the piezoelectric elements 28a to 28d are connected in common on the fiber holding member 29 by the piezoelectric elements 28a to 28d being mounted on a common connection wiring pattern 29a formed on the fiber holding member 29. The corresponding wiring cable 13 from the drive controller/resonance frequency detector 38 of the control device body 30 is connected to the power supply terminal (the other surface electrode) of the piezoelectric elements 28a to 28d. Similarly, a corresponding wiring cable 13 from the drive controller/resonance frequency detector 38 is connected to the connection wiring pattern 29a of the fiber holding member 29.

In this way, by connecting the ground terminals of the piezoelectric elements 28a to 28d to the driver 21 in common, five wiring cables 13 are sufficient for electrically connecting the piezoelectric elements 28a to 28d with the drive controller/resonance frequency detector 38. By contrast, when the ground terminals of the piezoelectric elements 28a to 28d are not connected in common, two wiring cables are required for each of the piezoelectric elements 28a to 28d, making it necessary to dispose a total of eight wiring cables inside the insertion part 23 of the scope 20. Accordingly, this embodiment allows a reduction in the number of wiring cables 13, with a corresponding reduction in size and diameter of the insertion part 23. Furthermore, the ground terminals of the piezoelectric elements 28a to 28d can be connected in common on the fiber holding member 29 by, for example, the piezoelectric elements 28a to 28d being mounted on the common connection wiring pattern 29a formed on the fiber holding member 29, thereby also simplifying the structure of the driver 21.

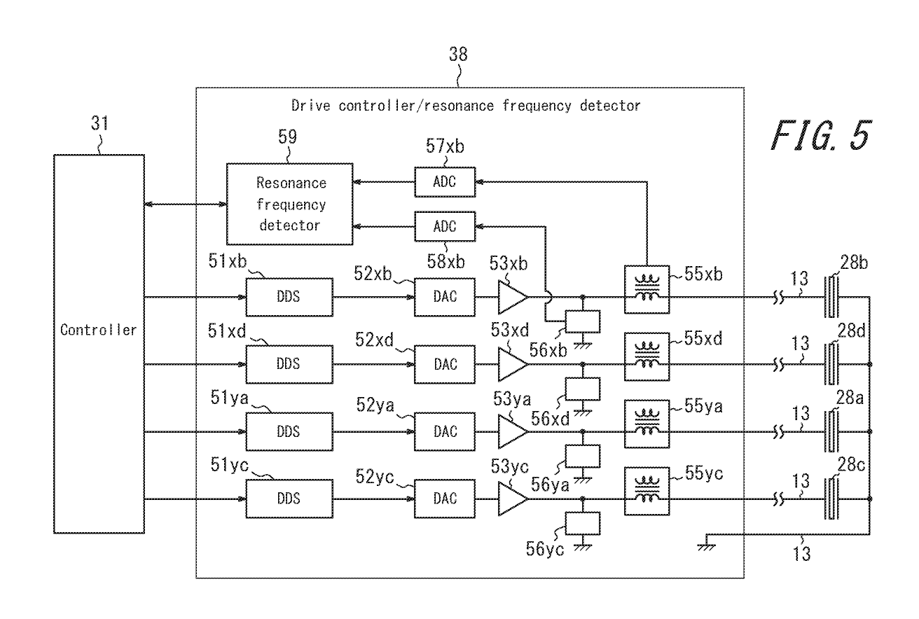

FIG. 5 is a block diagram schematically illustrating the structure of the drive controller/resonance frequency detector 38. The drive controller/resonance frequency detector 38 includes a Digital Direct Synthesis (DDS) transmitter 51ya, Digital/Analog Converter (DAC) 52ya, and amplifier 53ya corresponding to the piezoelectric element 28a, a DDS 51xb, DAC 52xb, and amplifier 53xb corresponding to the piezoelectric element 28b, a DDS 51yc, DAC 52yc, and amplifier 53yc corresponding to the piezoelectric element 28c, and a DDS 51xd, DAC 52xd, and amplifier 53xd corresponding to the piezoelectric element 28d. Unless otherwise specified, these components are collectively abbreviated as DDSes 51, DACs 52, and amplifiers 53. The DDSes 51 receive input of a corresponding control signal from the controller 31 and generate a digital driving signal. After each of these digital driving signals is converted to an analog signal by the corresponding DAC 52, the analog signal is amplified by the corresponding amplifier 53. Via the corresponding wiring cable 13, the output of the amplifiers 53 is applied to the corresponding one of the piezoelectric elements 28a to 28d positioned at the tip 24 of the scope 20. As a result, the piezoelectric elements 28a to 28d are driven by vibration.

Voltage of equivalent magnitude and opposite sign is applied across the piezoelectric elements 28b and 28d in the X direction. As a result, when one of the piezoelectric elements 28b and 28d extends and the other contracts, the fiber holding member 29 is flexed. Repeating this process causes the fiber holding member 29 to vibrate in the X direction. Similarly, voltage of equivalent magnitude and opposite sign is applied across the piezoelectric elements 28a and 28c in the Y direction, causing the fiber holding member 29 to vibrate in the Y direction.

The drive controller/resonance frequency detector 38 applies vibration voltage of the same frequency or vibration voltage of different frequencies to the piezoelectric elements 28b and 28d for driving in the X direction and the piezoelectric elements 28a and 28c for driving in the Y direction. Upon vibration driving of each of the piezoelectric elements 28a and 28c for driving in the Y direction and the piezoelectric elements 28b and 28d for driving in the X direction, the emission end 11b of the optical fiber 11 for illumination illustrated in FIG. 3, FIG. 4A, and FIG. 4B vibrate. As a result, the emission end face 11c is then selected, and the laser light emitted from the emission end face 11c sequentially scans the surface of the object 100.

The emission end 11b of the optical fiber 11 for illumination is subjected to vibration driving at the resonance frequency in one or both of the X direction and the Y direction. The resonance frequency of the emission end 11b, however, changes based on environmental conditions and also changes over time. Therefore, in this embodiment, the resonance frequency of the emission end 11b of the optical fiber 11 for illumination is detected in the drive controller/resonance frequency detector 38.

In FIG. 5, in order to detect the resonance frequency of the emission end 11b, the drive controller/resonance frequency detector 38 includes, at the power supply terminal side of the piezoelectric elements 28a to 28d, current detectors 55ya, 55xb, 55yc, and 55xd that detect the current flowing in the corresponding piezoelectric elements 28a to 28d, and voltage detectors 56ya, 56xb, 56yc, and 56xd that detect the applied voltage. Unless otherwise specified, these components are collectively abbreviated as current detectors 55 and voltage detectors 56. The current detectors 55 may be configured using Current Transformers (CTs). The current detectors 55 are not limited to CTs and may be configured with a known integrated circuit or the like. In particular, by using CTs, a low-voltage circuit configuration is possible even when the voltage applied to the corresponding piezoelectric element is a relatively high voltage, thus allowing a reduction in size and cost of the current detectors 55. Using CTs also allows the detection system to be arranged on the 2D circuit side, which offers the advantage of simplifying insulation from the patient circuit.

The drive controller/resonance frequency detector 38 further includes Analog/Digital Converters (ADCs), which convert, to a digital signal, the current and voltage detected by the current detectors 55 and voltage detectors 56 respectively corresponding to the piezoelectric elements 28a to 28d, and a resonance frequency detector 59 that detects the resonance frequency in the corresponding vibration direction from the phase difference in the current and voltage that were converted to digital signals. To simplify the drawing, FIG. 5 only shows the ADC 57xb corresponding to the current detector 55xb and the ADC 58xb corresponding to the voltage detector 56xb. The other ADCs are omitted from the drawing. Unless otherwise specified, these components are collectively abbreviated as ADCs 57 and ADCs 58. The output of the ADCs 57 and the ADCs 58 is also provided to the controller 31.

Through control by the controller 31, the ADCs 57 convert the output of the current detectors 55 to a digital signal at the time at which the amplitude of the current detected by the current detectors 55 reaches a maximum. Similarly, through control by the controller 31, the ADCs 58 convert the output of the voltage detectors 56 to a digital signal at the time at which the amplitude of the voltage reaches a maximum. As a result, the current and voltage can be detected with a high S/N ratio, thus allowing accurate driving control.

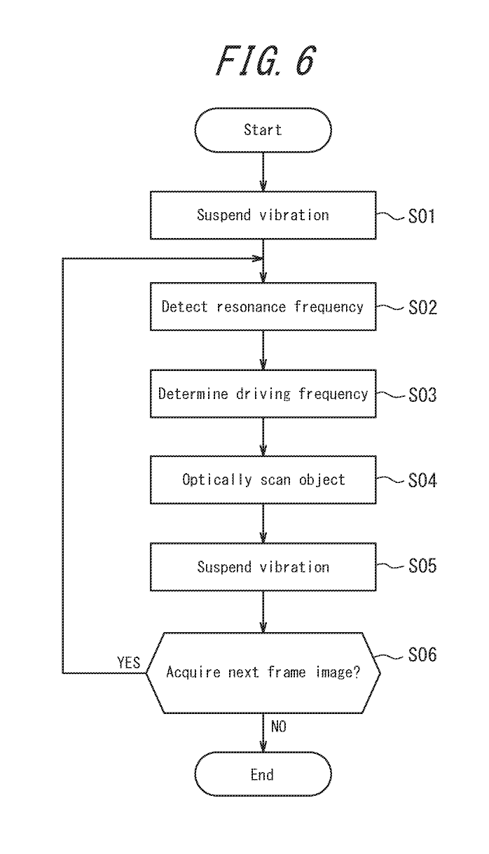

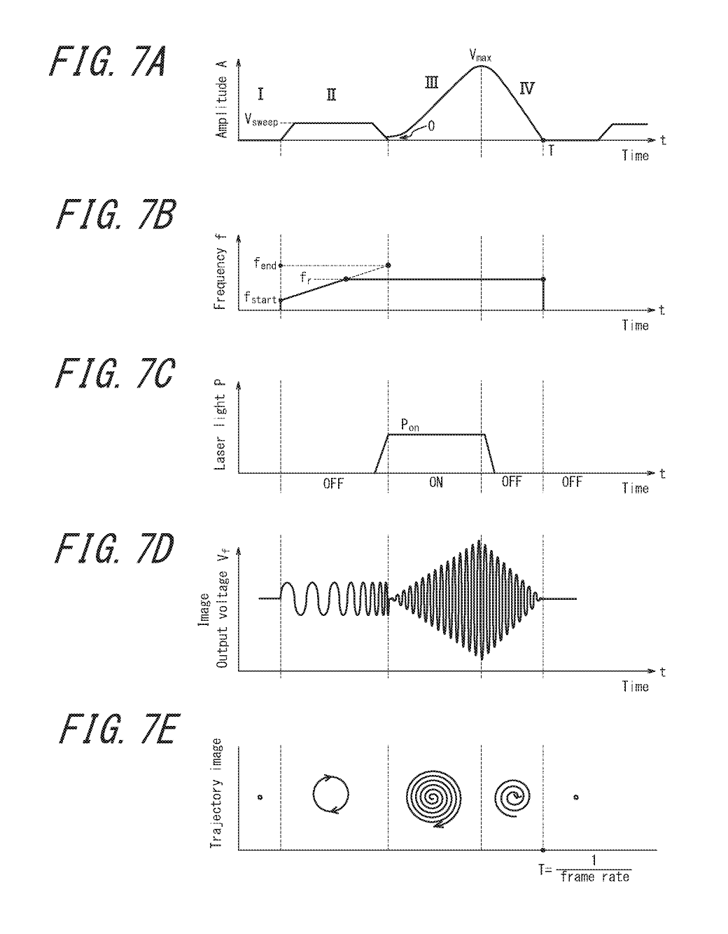

Operations of the optical scanning endoscope apparatus 10 are now described with reference to FIG. 6 and FIGS. 7A to 7E. FIG. 6 is a flowchart describing operations. FIGS. 7A to 7E illustrate the operation timing and the content of the operation of each component, along with the scanning trajectory of illumination light. FIG. 7A shows the amplitude A of driving voltage, FIG. 7B shows the frequency f of driving voltage, FIG. 7C shows laser output P of the lasers 33R, 33G, and 33B, FIG. 7D shows the waveform of output voltage Vf, and FIG. 7E shows the scanning trajectory of illumination light emitted from the optical fiber 11 for illumination. In FIGS. 7A to 7D, the horizontal axis t represents time.

First, the initial state is a state in which operation of the emission end 11b of the optical fiber 11 for illumination is suspended (step S01). This state is represented as period I in FIG. 7A.

Next, the controller 31 starts a resonance frequency detection step to detect the resonance frequency (step S02). The resonance frequency detection step corresponds to period II in FIG. 7A. In period II, vibration voltage with an amplitude A equivalent to a predetermined amplitude V_{sweep}, a phase that is shifted by 90° between the X and Y directions, and a frequency f that increases over time is applied to the piezoelectric elements 28b and 28d in the X direction and the piezoelectric elements 28a and 28c in the Y direction (see FIGS. 7A, 7B, and 7D). As a result, the vibration frequency of the emission end 11b of the optical fiber 11 for illumination is swept within a predetermined frequency range. The predetermined frequency range is predicted in advance as a range that is around the resonance frequency at the time of design and over which the resonance frequency can vary. At this time, the lasers 33R, 33G, and 33B are not yet turned on (FIG. 7C). As a result, the emission end face 11c of the optical fiber 11 for illumination vibrates so as to trace a circle (FIG. 7E).

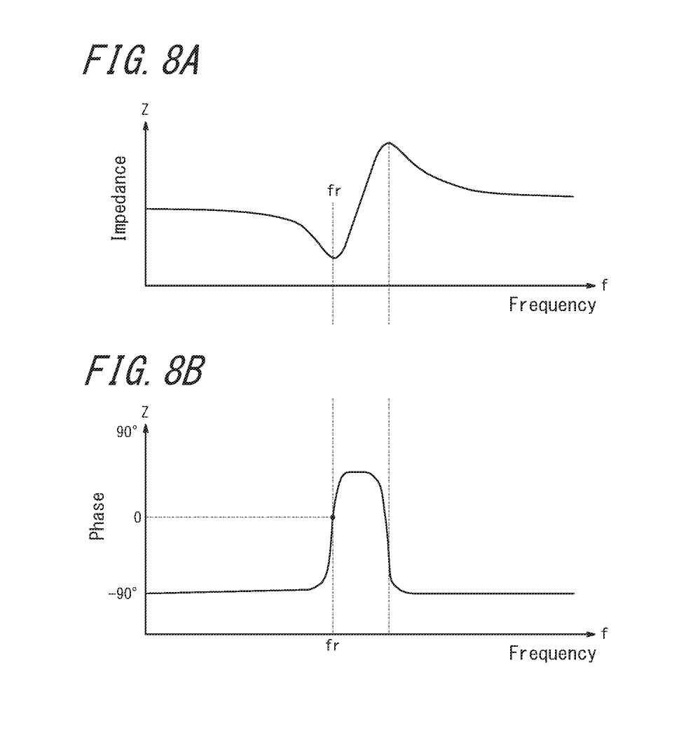

While the frequency of the driving voltage is increasing, the current signals and voltage signals detected by the corresponding current detectors 55 and voltage detectors 56 are monitored by the resonance frequency detector 59. The resonance frequency detector 59 detects the resonance frequency by detecting the shift in phase of the current signal and the voltage signal (the temporal shift of the maximum value of each signal). In general, the frequency characteristics of the vibration circuit's impedance and of the phase shift in current and voltage are known to be as in FIG. 8A and FIG. 8B. At the time of vibration at the resonance frequency, the impedance is minimized, and the phase shift is zero. The resonance frequency detector 59 identifies the frequency fr at the time that the phase shift of the current signal from the corresponding current detector 55 and the voltage signal from the corresponding voltage detector 56 is zero as being the resonance frequency and outputs the resonance frequency to the controller 31.

The controller 31 determines the subsequent driving frequency to be near the detected resonance frequency fr (step S03). The driving frequency allows driving at a frequency near the resonance frequency fr, but the driving frequency need not match fr exactly and may be a slightly different value. The driving frequency determination step to determine the driving frequency is performed during period II.

If the resonance frequency is not detected in the resonance frequency detection step (step S02), either there is no output from the resonance frequency detector 59 to the controller 31, or a signal detecting an error is transmitted. In this case, the controller 31 determines that an error has occurred, suspends the apparatus, and displays a warning indicating an error on the display 40. Possible examples of when the resonance frequency is not detected include the optical fiber 11 for illumination being broken and an error in the piezoelectric elements 28a to 28d.

Immediately before the end of period II, the controller 31 turns on the lasers 33R, 33G, and 33B. Next, as the scanning step, the object is optically scanned (step S04). In other words, in period III, the controller 31 fixes the driving frequency f of the voltage applied to the piezoelectric elements 28b and 28d in the X direction and the piezoelectric elements 28a and 28c in the Y direction at the resonance frequency fr (FIG. 7B) and increases the amplitude A of the driving voltage from zero to the maximum value Vmax over time (FIG. 7A). As a result, the light emitted from the optical fiber 11 for illumination follows a spiral trajectory in which the radius increases over time (FIG. 7E). At this time, based on output of the ADCs 57, the controller 31 performs feedback control so that the maximum value of the current detected by the current detectors 55 becomes constant. The controller 31 also monitors output of the ADCs 58.

Next, upon detecting that the amplitude A of the driving voltage output by the ADCs 58 has reached the maximum value Vmax, the controller 31 suspends oscillation of the lasers 33R, 33G, and 33B and also gradually suspends vibration of the optical fiber 11 for illumination (step S05). Vibration is suspended by rapidly decreasing the amplitude A of the driving voltage in period IV, which is shorter than period III. By the above-described spiral scanning, a circular region of the object 100 is scanned in 2D, and one frame of an image is acquired. In the case of acquiring the next frame, the controller 31 returns to step S02 again and repeats step S02 through step S05. Accordingly, in this embodiment, the controller 31 and the drive controller/resonance frequency detector 38 constitute a controller that controls the driver 21.

According to the optical scanning endoscope apparatus 10 of this embodiment, as described above, the insertion part 23 of the scope 20 can be reduced in size and diameter, the structure of the driver 21 can be simplified, the current detectors 55 can be reduced in size and cost, accuracy of current detection can be improved, and insulation from the patient circuit can be simplified, among other effects. The optical scanning endoscope apparatus 10 detects the resonance frequency fr before scanning the object 100 and acquires an image by optically scanning the object under observation at this resonance frequency fr. Therefore, it is possible to prevent a decrease in performance due to misalignment with the resonance frequency of the fiber resulting from variability between apparatuses and change over time, and the driving frequency can be adjusted appropriately. Always subjecting the emission end 11b of the optical fiber 11 for illumination to vibration driving at a frequency near the resonance frequency also allows scanning with good energy efficiency.

Furthermore, since the resonance frequency is detected before each image frame is acquired, the driving frequency can be adjusted to an appropriate value immediately if the resonance frequency changes for a reason such as a temperature increase during operation of the optical scanning endoscope apparatus 10. As a result, the emission end face 11c of the optical fiber 11 for illumination can be vibrated over a stable trajectory. It is thus expected that a more stable image can be acquired and displayed.

Furthermore, in the resonance frequency detection step (step S02), when the resonance frequency cannot be detected, the apparatus is suspended and a warning is issued, thereby allowing early detection of an error in the apparatus and preventing malfunction or increased damage.

Instead of detecting the resonance frequency and determining the driving frequency for the second time onward after scanning is suspended in period IV, these operations may be performed by sweeping the vibration frequency f around the resonance frequency and detecting the resonance frequency while vibration is being reduced during period IV (step S05). In this case, optical scanning can start immediately after suspension of vibration (step S03), which increases the frame rate and allows acquisition of a better image.

### Embodiment 2

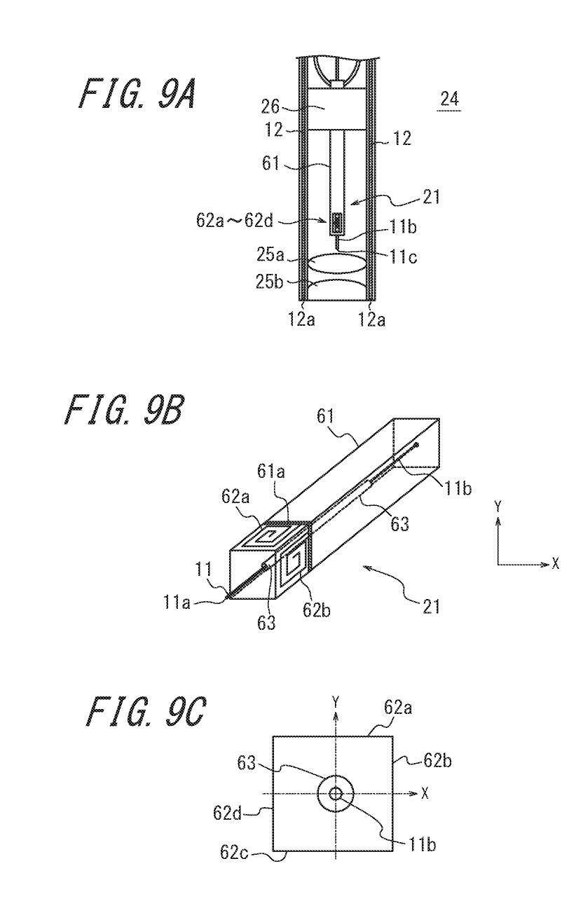

FIGS. 9A, 9B, and 9C are expanded diagrams illustrating the tip of the scope in the optical scanning endoscope apparatus according to Embodiment 2. This embodiment has the structure of the optical scanning endoscope apparatus 10 of Embodiment 1, except that instead of piezoelectric elements, the driver 21 is configured using a permanent magnet 63 fixed to the optical fiber 11 for illumination and coils 62a to 62d for generation of a deflecting magnetic field (electromagnetic coils) that drive the permanent magnet 63. Portions identical to the structure described in Embodiment 1 are labeled with the same reference signs, and a description thereof is omitted. The differences from Embodiment 1 are described below. FIG. 9A is a cross-sectional diagram of the tip 24 of the scope 20, FIG. 9B is an enlarged perspective view of the driver 21 in FIG. 9A, and FIG. 9C is a cross-sectional view perpendicular to the axis of the optical fiber 11 for illumination, illustrating a portion including the coils 62a to 62d for generation of a deflecting magnetic field and the permanent magnet 63 in FIG. 9B.

At a portion of the emission end 11b of the optical fiber 11 for illumination, the permanent magnet 63, which is magnetized in the axial direction of the optical fiber 11 for illumination and includes a through-hole, is joined to the optical fiber 11 for illumination by the optical fiber 11 being passed through the through-hole. A square tube 61, one end of which is fixed to the attachment ring 26, is provided so as to surround the emission end 11b, and flat coils 62a to 62d for generation of a deflecting magnetic field are provided on the sides of the square tube 61 at a portion thereof opposing one pole of the permanent magnet 63.

The pair of coils 62a and 62c for generation of a deflecting magnetic field in the Y direction and the pair of coils 62b and 62d for generation of a deflecting magnetic field in the X direction are each disposed on opposing sides of the square tube 61, and a line connecting the center of the coil 62a for generation of a deflecting magnetic field with the center of the coil 62c for generation of a deflecting magnetic field is orthogonal to a line connecting the center of the coil 62b for generation of a deflecting magnetic field with the center of the coil 62d for generation of a deflecting magnetic field near the central axis of the square tube 61 when the optical fiber 11 for illumination is disposed therein at rest.

The coils 62a to 62d for generation of a deflecting magnetic field each constitute a vibration element, and the ground terminals (one of the surface electrodes) of the coils are connected in common in the driver 21. For example, the ground terminals of the coils 62a to 62d for generation of a deflecting magnetic field are connected in common on the square tube 61 by the ground terminals being adhered to a common connection wiring pattern 61a formed on the square tube 61. The corresponding wiring cable 13 from the drive controller/resonance frequency detector 38 of the control device body 30 is connected to the power supply terminal (the other end) of each of the coils 62a to 62d for generation of a deflecting magnetic field. Similarly, a corresponding wiring cable 13 from the drive controller/resonance frequency detector 38 is connected to the connection wiring pattern 61a of the square tube 61. In this way, the driving current from the drive controller/resonance frequency detector 38 is supplied to the coils 62a to 62d for generation of a deflecting magnetic field, and due to electromagnetic action with the permanent magnet 63, the emission end 11b of the optical fiber 11 for illumination is vibrated.

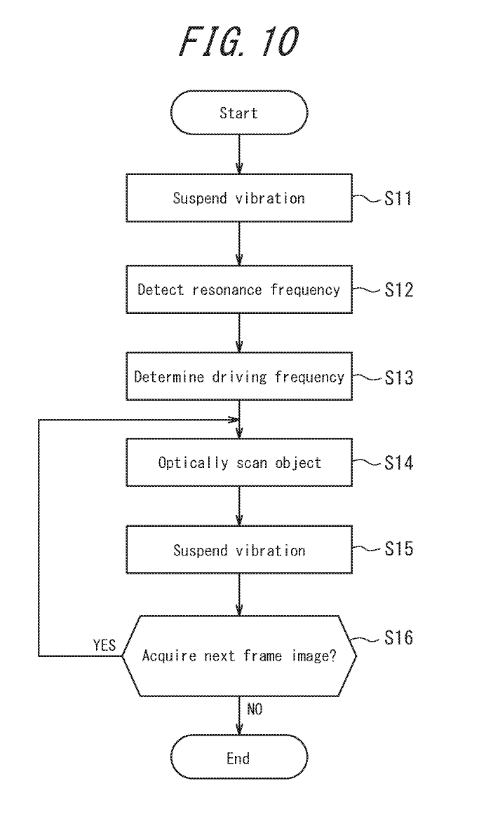

FIG. 10 is a flowchart illustrating operations of the optical scanning endoscope apparatus 10 according to this embodiment. Since the content of the steps in FIG. 10 is nearly the same as that of the steps in Embodiment 1, the steps in FIG. 10 are numbered by adding 10 to the reference numeral of the corresponding steps in FIG. 6. In this embodiment, however, after operation of the apparatus begins, the resonance frequency is detected only once (step S12), and the driving frequency is determined (step S13). Subsequently, acquisition of image data by optically scanning the object (step S14) is repeated until the controller 31 suspends acquisition of the next frame (step S16). Since the remaining structure and operations are similar to those of Embodiment 1, identical or corresponding constituent elements are labeled with the same reference signs, and a description thereof is omitted.

According to this embodiment, the number of wiring cables 13 for electrically connecting the coils 62a to 62d for generation of a deflecting magnetic field with the drive controller/resonance frequency detector 38 can be reduced, thereby obtaining the same effects as in Embodiment 1, such as a reduction in size and diameter of the insertion part 23 of the scope 20 and simplification of the structure of the driver 21. Furthermore, after detecting the resonance frequency once, image frames are acquired by repeated optical scanning. Therefore, endoscope images can be acquired at a higher frame rate than in Embodiment 1. In this embodiment, since the vibration elements are formed by coils, the current detectors 55 are not limited to current transformers, and a variety of known current sensors may be used.

### Embodiment 3

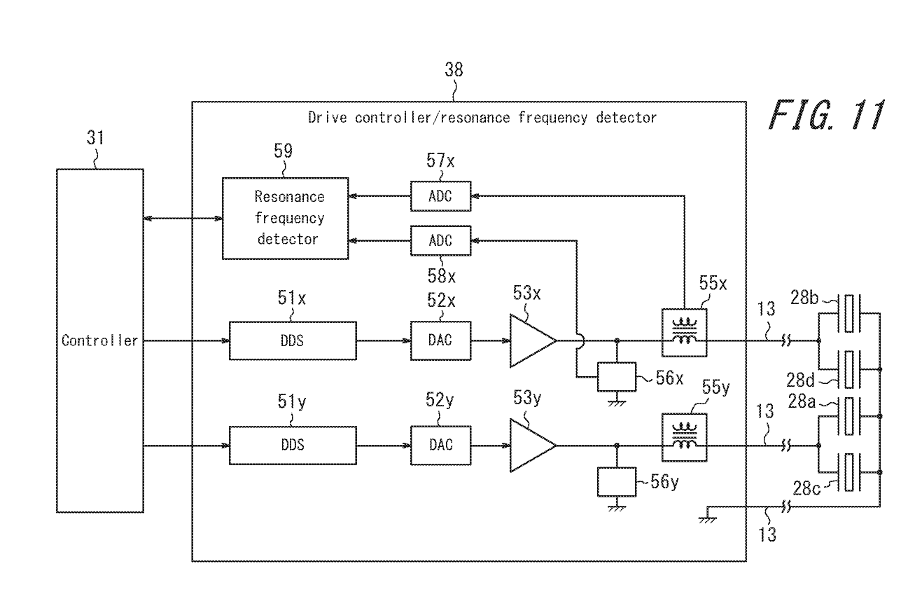

FIG. 11 is a block diagram schematically illustrating the main structure of an optical scanning endoscope apparatus according to Embodiment 3. This embodiment has the structure of the optical scanning endoscope apparatus 10 of Embodiment 1, except that the power supply terminals of the piezoelectric elements (first vibration element) 28a and 28c that vibrate the emission end 11b of the optical fiber 11 for illumination in the Y direction and the power supply terminals of the piezoelectric elements (second vibration element) 28b and 28d that vibrate the emission end 11b in the X direction are respectively connected in parallel at the driver 21 side. Portions identical to the structure described in Embodiment 1 are labeled with the same reference signs, and a description thereof is omitted. The differences from Embodiment 1 are described below.

The same driving signal is applied to the piezoelectric elements 28a and 28c via the corresponding DDS 51y, DAC 52y, amplifier 53y, and wiring cable 13. Similarly, the same driving signal is applied to the piezoelectric elements 28b and 28d via the corresponding DDS 51x, DAC 52x, amplifier 53x, and wiring cable 13. The piezoelectric elements 28a and 28c that form a pair are configured so that when the applied driving signal has a certain polarity, a first one of the piezoelectric elements 28a and 28c expands and a second one of the piezoelectric elements 28a and 28c contracts, whereas when the driving signal has the opposite polarity, the second one of the piezoelectric elements 28a and 28c expands and the first one of the piezoelectric elements 28a and 28c contracts. Similarly, the piezoelectric elements 28b and 28d that form a pair are configured so that when the applied driving signal has a certain polarity, a first one of the piezoelectric elements 28b and 28d expands and a second one of the piezoelectric elements 28b and 28d contracts, whereas when the driving signal has the opposite polarity, the second one of the piezoelectric elements 28b and 28d expands and the first one of the piezoelectric elements 28b and 28d contracts. As a result, the piezoelectric elements 28a to 28d are driven by vibration.

The combined current flowing in the piezoelectric elements 28a and 28c is detected by the current detector 55y, which is provided with a Current Transformer (CT), is converted to a digital signal by the ADC 57y, and is input into the resonance frequency detector 59. Similarly, the combined current flowing in the piezoelectric elements 28b and 28d is detected by the current detector 55x, which is provided with a Current Transformer (CT), is converted to a digital signal by the ADC 57x, and is input into the resonance frequency detector 59. The vibration voltage applied to the piezoelectric elements 28a and 28c is detected by the voltage detector 56y, is converted to a digital signal by the ADC 58y, and is input into the resonance frequency detector 59. Similarly, the vibration voltage applied to the piezoelectric elements 28b and 28d is detected by the voltage detector 56x, is converted to a digital signal by the ADC 58x, and is input into the resonance frequency detector 59. The output of the ADCs 57x and 57y and of the ADCs 58x and 58y is also provided to the controller 31. To simplify the drawing, FIG. 11 only shows the ADC 57x corresponding to the current detector 55x and the ADC 58x corresponding to the voltage detector 56x. The other ADCs are omitted from the drawing. The remaining structure and operations are similar to those of Embodiment 1.

According to this embodiment, in the driver 21, the ground terminals of the piezoelectric elements 28a to 28d are connected in common, and the power supply terminals of the piezoelectric elements 28b and 28d forming a pair in the X direction and the power supply terminals of the piezoelectric elements 28a and 28c forming a pair in the Y direction are respectively connected in parallel. Accordingly, the total number of wiring cables 13 for electrically connecting the piezoelectric elements 28a to 28d with the drive controller/resonance frequency detector 38 is reduced to three. Hence, the number of wiring cables 13 can be reduced beyond the number in Embodiment 1, which is advantageous in allowing a further reduction in size and diameter of the insertion part 23 of the scope 20.

This disclosure is not limited only to the above embodiments, and a variety of changes or modifications may be made. For example, the optical scanning is not limited to being spiral scanning and may instead be raster scanning. In this case, the optical fiber for illumination is only vibrated at the resonance frequency in one of the XY scanning directions. Furthermore, the vibration driving means is not limited to a method using coils and a magnet or a method using piezoelectric elements. Any other vibration driving means may be used. In the case of using coils, two coils in series may constitute one vibration element. Furthermore, the resonance frequency is not limited to being detected upon each scanning or at the start of driving the apparatus and may instead be detected at various timings. For example, possible settings include one detection per a plurality of scans, one detection per day, or detection upon user instruction. This disclosure is not limited to an endoscope apparatus and may also be adapted for use in another apparatus such as a microscope or a projector.

### REFERENCE SIGNS LIST

10 Optical scanning endoscope apparatus

11 Optical fiber for illumination

11b Emission end

21 Driver

28a to 28d Piezoelectric element

29a Connection wiring pattern

31 Controller

33R, 33G, 33B Laser

35 Photodetector

37 Image processor

38 Drive controller/resonance frequency detector

55 Current detector

61a Connection wiring pattern

62a to 62d Coil for generation of a deflecting magnetic field

63 Permanent magnet

100 Object

1. An optical scanning apparatus comprising:
an optical fiber;
a driver configured to drive an emission end of the optical fiber;
a current detector configured to detect a current flowing in the driver; and
a controller configured to scan light emitted from the optical fiber by controlling the driver based on output of the current detector; wherein
the driver comprises a plurality of vibration elements;
ground terminals of the vibration elements are connected in common to the driver; and
the current detector detects the current at a power supply terminal of the vibration elements.

2. The optical scanning apparatus of claim 1, wherein the current detector comprises a current transformer.

3. The optical scanning apparatus of claim 1, wherein
the vibration elements comprise a plurality of first vibration elements that vibrate the emission end in a first direction and a plurality of second vibration elements that vibrate the emission end in a second direction different from the first direction;
power supply terminals of the first vibration elements are connected in common to the driver; and
power supply terminals of the second vibration elements are connected in common to the driver.

4. The optical scanning apparatus of claim 1, wherein the controller controls the driver based on output of the current detector at a time when an amplitude of the current is maximized.

5. The optical scanning apparatus of claim 1, wherein the controller controls the driver so that a maximum value of the current detected by the current detector becomes constant.

6. An optical scanning observation apparatus comprising:
the optical scanning apparatus of claim 1;
a photodetector configured to detect light obtained from an object by optical scanning with the optical scanning apparatus and to convert the light to an electrical signal; and
an image processor configured to generate an image based on the electrical signal output from the photodetector.
